(19) United States
(12) Patent Application Publication
(10) Pub. No.: US 2007/0073209 A1
(43) Pub. Date: Mar. 29, 2007

(54) Skin closure device

(76) Inventors: Prasanta K. Raj, Bay Village, OH (US); Joyesh Raj, Bay Village, OH (US)

Correspondence Address: TAROLLI, SUNDHEIM, COVELL & TUMMINO L.L.P., 1300 EAST NINTH STREET, SUITE 1700, CLEVEVLAND, OH, 44114, US

(21) Appl. No.: 11/235,756

(22) Filed: Sep. 27, 2005

(51) Int. Cl. A61F 15/00

(52) U.S. Cl. 602/42

(57) ABSTRACT

A skin closure device is used in closing a circuitous opening in the skin of a patient, such as a wound or incision. The opening is defined by circuitous lateral sides extending indirectly from a first end to a second end of the opening along a length of the opening. The lateral sides define a predetermined shape of the opening. The skin closure device includes an adhesive strip having a first end portion, a second opposite end portion, and a circuitous central portion extending indirectly from the first end portion to the second end portion. The first end portion covers the first end of the opening. The second end portion covers the second end of the opening. The central portion is engageable with the lateral sides defining the opening. The adhesive strip has a predetermined shape substantially corresponding to the predetermined shape of the opening. Accordingly, closure devices having desired circuitous shapes may be provided during a surgical procedure in which a desired circuitous incision is made, such as during a peri-areolar breast biopsy.

### FIELD OF THE INVENTION

The present invention relates to a skin closure device, more specifically, the present invention relates to a skin closure device for closing a circuitous opening in the skin of a patient.

### BACKGROUND OF THE INVENTION

Medical adhesive tapes, surgical tapes, or adhesive strips, such as Steri-strips® manufactured by 3M®, are used to help hold an opening in the skin, such as a wound or incision, closed. The adhesive strips are linear. The adhesive strips are placed on the skin with the length of the strips extending across the length of the wound or incision to approximate the wound margins. The wound margins may separate upon removal of the adhesive strips. It has also been known to place adhesive strips with the length of the strips extending at least partially along the length of the wound or incision to help prevent separation of the wound margins during removal of the adhesive strips. If the wound or incision is circuitous or not in a straight line, a plurality of adhesive strips must be placed along the length of the wound or incision.

### SUMMARY OF THE INVENTION

The present application is directed to a skin closure device for use in closing a circuitous opening in the skin of a patient, such as a wound or incision. The opening is defined by circuitous lateral sides extending indirectly from a first end to a second end of the opening along a length of the opening. The lateral sides define a predetermined shape of the opening. The skin closure device includes an adhesive strip having a first end portion, a second opposite end portion, and a circuitous central portion extending indirectly from the first end portion to the second end portion. The first end portion covers the first end of the opening. The second end portion covers the second end of the opening. The central portion is engageable with the lateral sides defining the opening. The adhesive strip has a predetermined shape substantially corresponding to the predetermined shape of the opening. Accordingly, closure devices having desired circuitous shapes may be provided during a surgical procedure in which a desired circuitous incision is made, such as during a peri-areolar breast biopsy.

### BRIEF DESCRIPTION OF THE DRAWINGS

The foregoing and other features of the present invention will become apparent to one skilled in the art to which the present invention relates upon consideration of the following description of the invention with reference to the accompanying drawings, in which:

FIG. 1 is a schematic view of a first embodiment of a skin closure device shown closing a circuitous opening in the skin of a patient;

FIG. 2 is a schematic view of a second embodiment of a skin closure device; and

FIG. 3 is a schematic view of the skin closure device of FIG. 2 shown closing a circuitous opening around an areola of a patient.

### DESCRIPTION OF THE INVENTION

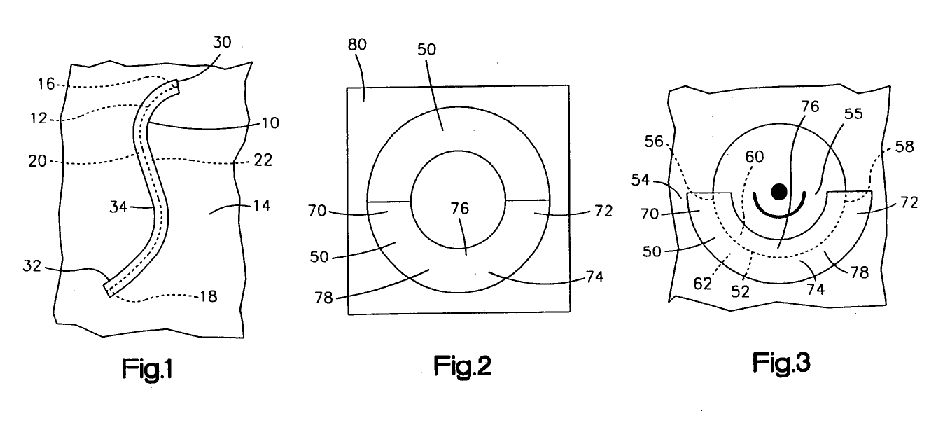

The present invention relates to a skin closure device for use in closing a circuitous opening in the skin of a patient, such as a wound or an incision. FIG. 1 illustrates a skin closure device 10, constructed in accordance with a first embodiment, for use in closing a circuitous opening 12 in the skin 14 of a patient. The opening 12 may be a wound or an incision.

The opening 12 has a first end 16 and a second end 18. The opening is defined by circuitous lateral sides 20 and 22 extending indirectly from the first end 16 to the second end 18 along a length of the opening 12. Accordingly, the lateral sides 20 and 22 do not extend in straight lines from the first end 16 to the second end 18. The lateral sides 20 and 22 extend generally parallel to each other. The lateral sides 20 and 22 define a predetermined circuitous shape of the opening 12. The lateral sides 20 and 22 define a curvilinear shape in FIG. 1, however, it is contemplated that the lateral sides 20 and 22 may define any desired circuitous shape.

The skin closure device 10 may be a medical adhesive tape, surgical tape, or an adhesive strip. The skin closure device 10 may include a pressure sensitive adhesive on a porous or non-porous backing. The pressure sensitive material may be on the entire length of the skin closure device 10. The skin closure device 10 helps approximate the lateral sides 20 and 22 or wound margins of the opening 12.

The adhesive strip 10 has a first end portion 30, a second opposite end portion 32, and a circuitous central portion 34 extending indirectly from the first end portion to the second end portion. Accordingly, the central portion 34 does not extend in a straight line. The first end portion 30 covers the first end 16 of the opening 12. The second end portion 32 covers the second end 18 of the opening 12. The central portion 34 is engageable with the lateral sides 20 and 22 defining the opening 12 to approximate the lateral sides.

The adhesive strip 10 has a predetermined shape substantially corresponding to the predetermined shape of the opening 12. The adhesive strip 10 has a curvilinear shape that substantially corresponds to the curvilinear shape of the opening 12, as shown in FIG. 1. The adhesive strip 10 may have any desired circuitous shape substantially corresponding to the predetermined circuitous shape of the opening 12.

During a surgical procedure, an incision or opening 12 in the skin 14 of the patient is made. The incision or opening 12 has a predetermined shape that the surgeon repeats each time the surgeon performs the procedure. Accordingly, the skin closure device 10 may have a predetermined circuitous shape substantially corresponding to the predetermined shape of the incision that the surgeon repeats.

As representative of a second embodiment of the present invention, FIGS. 2 and 3 schematically illustrate a skin closure device 50 for use during a peri-areolar breast biopsy. The skin closure device 50 (FIG. 3) may be used to help close a circuitous opening 52 in the skin 54 of a patient. The opening 52 may be an incision that extends at least partially around the areola 55 of the patient.

The opening 52 has a first end 56 and a second end 58. The opening 52 is defined by circuitous lateral sides 60 and 62 extending indirectly from the first end 56 to the second end 58 along a length of the opening 52. Accordingly, the lateral sides 60 and 62 do not extend in straight lines from the first end 56 to the second end 58. The lateral side 60 may include a portion of the areola 55. The lateral sides 60 and 62 extend generally parallel to each other. The lateral sides 60 and 62 define a predetermined circuitous shape of the opening 52. The lateral sides 60 and 62 define an arcuate shape in FIG. 3. It is contemplated that the opening 52 may extend any desired distance around the areola 55.

The skin closure device 50 may be a medical adhesive tape, surgical tape, or an adhesive strip. The skin closure device 50 may include a pressure sensitive adhesive on a porous or non-porous backing. The pressure sensitive material may be on the entire length of the skin closure device 50. The skin closure device 50 helps approximate the lateral sides 60 and 62 or wound margins of the opening 52.

The adhesive strip 50 has a first end portion 70, a second opposite end portion 72, and a circuitous central portion 74 extending indirectly from the first end portion to the second end portion. The first end portion 70 covers the first end 56 of the opening 52. The second end portion 72 covers the second end 58 of the opening 52. The central portion 74 is engageable with the lateral sides 60 and 62 defining the opening 52. The adhesive strip 50 has a predetermined shape substantially corresponding to the predetermined shape of the opening 52. The adhesive strip 50 has an arcuate shape that corresponds to the shape of the opening 52.

The central portion 74 of the adhesive strip 50 has a radially inner portion 76 that engages the lateral side 60 defining the opening 52. The central portion 74 has a radially outer portion 78 that engages the lateral side 62 defining the opening 52. The radially inner portion 76 and the radially outer portion 78 extend indirectly from the first end portion 70 to the second end portion 72.

The adhesive strip 50 (FIG. 2) may be carried on a protective liner 80. The adhesive strip 50 is removed from the liner 80 for applying to the skin 54 of the patient. A plurality of adhesive strips 50 may be carried on the liner 80. FIG. 2 shows two adhesive strips 50 carried on the liner 80. It is contemplated that the plurality of strips 50 may form a full circle on the liner 80. Any desired number of adhesive strips 50 may be provided to form the circle. It is also contemplated that a one piece annular adhesive strip 50 may be provided on the liner 80. The annular adhesive strip 50 may be cut to any desired length that substantially corresponds to the length of the opening 52 formed around the areola 55.

It is contemplated that the adhesive strip or strips 50 may be provided with an outer diameter of 50 mm, 58 mm, or 66 mm to be used for areola having diameters of 38 mm, 42 mm, or 50 mm. The adhesive strip or strips 50 may have an inner diameter of 26 mm or 34 mm. Accordingly, the adhesive strip 50 may engage the areola 55 and the skin adjacent the areola.

From the above description of the invention, those skilled in the art will perceive improvements, changes and modifications. Such improvements, changes and modifications within the skill of the art are intended to be covered by the appended claims.

Having described the invention, we claim:

1. A skin closure device for use in closing an opening in the skin of a patient, the opening being defined by circuitous lateral sides extending indirectly from a first end to a second end of the opening along a length of the opening, the lateral sides defining a predetermined shape of the opening, said skin closure device comprising an adhesive strip having a first end portion, a second opposite end portion, and a circuitous central portion extending indirectly from said first end portion to said second end portion, said first end portion being configured to cover the first end of the opening, said second end portion being configured to cover the second end of the opening, and said central portion being engageable with the lateral sides defining the opening, said adhesive strip having a predetermined shape substantially corresponding to the predetermined shape of the opening.

2. A skin closure device as set forth in claim 1 wherein said predetermined shape is curvilinear.

3. A skin closure device as set forth in claim 1 wherein said adhesive strip has a length substantially corresponding to the length of the opening.

4. A skin closure device as set forth in claim 1 wherein said predetermined shape is arcuate.

5. A skin closure device as set forth in claim 4 wherein the opening extends around a portion of the areola of the patient, said skin closure device being engageable with the areola and the skin adjacent the areola.

6. A skin closure device as set forth in claim 4 wherein said skin closure device has an outer diameter larger than an outer a diameter of the areola and an inner diameter smaller than the other diameter of the areola.

7. A skin closure device as set forth in claim 4 further including a protective liner which carries said skin closure device prior to application of said skin closure device to the skin, said skin closure device comprising a plurality of strips carried on said liner.

8. A skin closure device as set forth in claim 1 wherein said predetermined shape is annular.

9. A skin closure device as set forth in claim 1 including a pressure sensitive adhesive on the entire length of said skin closure device.
